(19) United States
(12) Patent Application Publication
(10) Pub. No.: US 2010/0016990 A1
(43) Pub. Date: Jan. 21, 2010

(54) Microelectromechanical system (MEMS) employing wireless transmission for providing sensory signals

(75) Inventor: Anthony D. Kurtz, Saddle River, NJ (US)

Correspondence Address: TROUTMAN SANDERS LLP;BANK OF AMERICA PLAZA, 600 PEACHTREE STREET, N.E., SUITE 5200, ATLANTA, GA, 30308-2216, US

(73) Assignee: Kulite Semiconductor Products, Inc., Leonia, NJ (US)

(21) Appl. No.: 12/220,012

(22) Filed: Jul. 21, 2008

(51) Int. Cl. A61F 2/48

(52) U.S. Cl. 623/24

(57) ABSTRACT

A medical system employs wireless transmission and provides sensory signals to a user of a prosthetic or other medical device. A series of pressure, force or strain sensors are placed upon various areas of the prosthetic device. The sensors are strategically placed according to anticipated functions of the prosthetic device and the sensors may be placed in clusters, where each cluster may include more than one sensor. The prosthetic device is normally operated by a biometric controller. The biometric controller is controlled by the handicapped user via muscles or other devices to enable the prosthetic device to perform various desired functions. During performing of such functions, the sensors will respond and produce outputs according to applied pressure or strain. These voltage outputs are transmitted by a transmitter to a remote receiver which is located on the body or person of the handicapped user. The receiver demodulates the transmitted signal to provide output signals proportional to the sensor signals as transmitted. These output signals are then directed to electrodes, probes or terminal pads imbedded in the body of the handicapped user by a physician or suitable technician. The imbedded probes or electrodes receive the sensor signals from the receiver and operate to stimulate the nerves so that a user can receive signals indicative of the force applied to given areas of the prosthetic device. In this manner the user can better control prosthetic device operation.

### FIELD OF THE INVENTION

This invention relates to medical devices and more particularly to a wireless system capable of responding to the operation of prosthetic devices or other medical implants to provide sensory outputs.

### BACKGROUND OF THE INVENTION

Prosthetic devices have been employed for many years. Such devices are typically controlled by a biometric controller, which controller is controlled by activation of certain muscles concerning the user. In this manner the user contracts or operates certain muscles, which muscles or other devices send signals to the biometric controller and the biometric controller produces output signals to control the motors or other devices controlling various parts of the prosthetic device. Apart from direct control of the prosthetic device, there are numerous techniques in the prior art for providing feedback signals to the user, which feedback signals emanate from the prosthetic device to give the user some indication of the pressure applied by the prosthetic device to the surface of an object. For example, a user of a prosthetic hand or a prosthetic claw would not wish to apply as much pressure to a crystal glass as he would apply to a plastic glass. It is of course understood that it would be desirable to give the user some indication of how much pressure is being applied by the prosthetic device. While the prosthetic device may be a hand or prosthetic claw it can of course be any other device such as a prosthetic foot and so on. Essentially, as indicated, the prior art was cognizant of such problems and indicated that the problems exist.

Reference is made to U.S. Pat. No. 5,480,454 issued on Jan. 2, 1996 entitled Control System for Prosthetic Devices to Bozeman, Jr., this Patent shows a control system and method for prosthetic devices. Basically the control system uses a transducer for receiving movement from a body part and for generating a sensing signal associated with that movement. Eventually command signals are provided which command signals are used for driving the prosthesis device and sub-prosthesis devices such as for example, it may control finger and wrist motion and related pressures. The control is determined by a typical harness or a shoulder controlled hardware which the handicapped person would use to control the prosthesis. As seen from that Patent there are parts of the prosthetic device that can be controlled and command signals are generated to produce such control. In any event, the Patent does not show feedback means for producing a signal back to the user as to the extent of such control.

Reference is made to U.S. Pat. No. 6,344,062 issued on Feb. 5, 2002 entitled Biometric Controller for a Multi-Finger Prosthesis. That Patent discloses a control system for use with a prosthetic device. The control system provides a control signal indicative of the desired movement of a body part. That system is also a control system for use with a prosthetic or orthotic device and uses a pneumatic sensor for sensing movement of a muscle, tendon or ligament intended to cause an associated movement of another body part and means for analyzing the signal and sending control signals. Essentially, as one can see the system is operative to control a prosthesis or to control digits such as fingers in hand in restoration operations and describes a biometric controller.

U.S. Pat. No. 6,500,210 issued on Dec. 31, 2002 entitled System and Method for Providing a Sense of Feel in a Prosthetic or Sensory Impaired Limb. This patent shows apparatus for providing a stimuli to a person having a prosthetic foot. Essentially, it uses an electronic circuit to control a vibrating motor which produces vibrations according to the pressure applied by the foot on a corresponding surface. This, as one can ascertain is an attempt to give the user a sensory feedback so that he can better control the foot.

U.S. Pat. No. 6,701,189 issued on Mar. 2, 2004 entitled Systems and Methods for Performing Prosthetic or Therapeutic Neuromuscular Stimulation Using a Universal External Controller Accommodating Different Control Inputs and/or Different Control Outputs. As one can see from the above noted Patent the systems and methods provide neuromuscular simulation for different prosthetic devices. Essentially the system describes a controller which is a biometric controller which receives control input signals from the user which input signals may come from shoulders or various other biological parts of the user to generate control signals for the prosthetic device and thus is designated as biometric controller.

One can view Patent Application No. US 2004/0146235 published on Jul. 29, 2004 entitled Process to Create Artificial Nerves for Biomechanical Systems Using Optical Waveguide Network. This system outlines the development of in-line distributed optical fiber microsensors for monitoring dynamic strain which is converted into touch and feel sensations in large and small artificial prosthetic devices.

U.S. Pat. No. 7,029,500 issued on Apr. 18, 2006 entitled Electronically Controlled Prosthetic System, this patent shows a prosthetic joint system for an artificial foot which essentially controls the foot by producing control signals regarding the load on the heel and toe as well angle sensors. These control signals serve to operate motors associated with the prosthetic device to provide improved control.

Reference is also made to Re-issue 39,961 reissued Dec. 25, 2007 entitled Computer Controlled Hydraulic Resistance Device for a Prosthesis and Other Apparatus. This device produces controlled signals for a prosthetic knee and operates to control the knee via such signals. The above noted patents as cited are incorporated herein in their entirety and essentially were cited by the applicant to show that there exists a number of innovations made to prosthetic devices. One such innovation involves controllers or devices which control the prosthetic device via inputs made by the user using various other muscles which are functional to produce signals to control the prosthetic device according to desired movements of the users such biometric controllers, as evidenced by the prior art, are well known. Still other devices respond to sensory signals produced by a prosthetic device while it is being operated. These signals, for example, are now coupled to motors or other devices as well as coupled to nerves or muscles to attempt to provide the user with additional information regarding operation of such devices. In any event, it is the object of the present invention to provide an improved system for providing sensory signals from a prosthetic device to a user, to enable a user to receive information regarding the various portions of the prosthetic device being operated and to enable selection of such portions or to select various other portions of the prosthetic device to provide sensory feedback.

It is a main object of the present invention to provide a wireless transmission where the sensory signals are processed by a transmitter and sent via a wireless link to a receiver, which receiver produces output signals indicative of stress or pressure imposed on various parts of the prosthetic device to enable the user to control the device with more accuracy because of such signals.

### SUMMARY OF THE INVENTION

A medical system for providing sensory signals to a user indicative of the force or pressure applied by a medical device controlled by the user, comprising: a plurality of force responsive sensors positioned on said device in predetermined locations, each of said devices operative to provide an output signal proportional to said applied force, scanning means scanning each of said sensors to provide a plurality of time sequential output signals each indicative of an associated sensor output, and a transmitter means coupled to said scanning means and operative to transmit a signal to a remote location indicative of said output signal of at least a first selected plurality of said force responsive sensors.

### BRIEF DESCRIPTION OF THE FIGURES

FIG. 1 is a schematic diagram of a prosthetic control system employing wireless transmission of sensory signals according to this invention.

FIG. 2 consist of FIGS. 2A and 2B and shows a piezoresistive semiconductor device which can be employed in conjunction with this invention.

FIG. 3 is a logic diagram showing the selection and positioning of various sensors on prosthetic hand.

FIG. 4 is a block diagram of a typical transmitter according to this invention. FIG. 4A is a diagram showing a typical transmitted signal obtained at the output of the transmitter.

FIG. 5 is a block diagram of the input to a modulator according to this invention.

FIG. 6 is a logic diagram showing operation of the microprocessor in the transmitter according to this invention.

FIG. 7 is a block diagram depicting additional functions the transmitter microprocessor can perform.

FIG. 8 is a block diagram of a receiver according to this invention.

FIG. 9 is a block diagram depicting logic performed by the microprocessor and the receiver.

FIG. 10 is a block diagram depicting the demodulator and processor utilized in the receiver according to this invention.

### DETAILED DESCRIPTION OF THE FIGURES

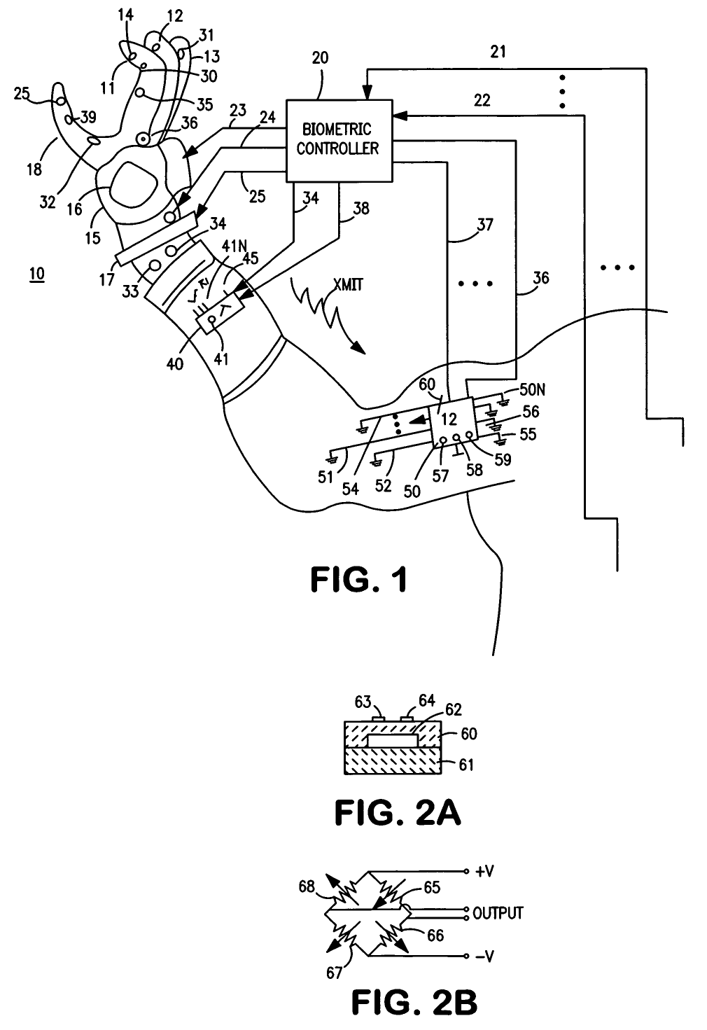

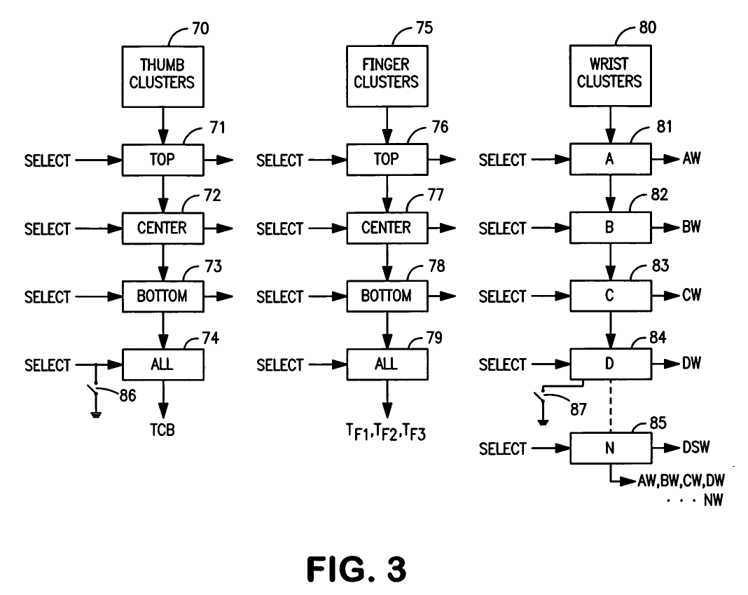

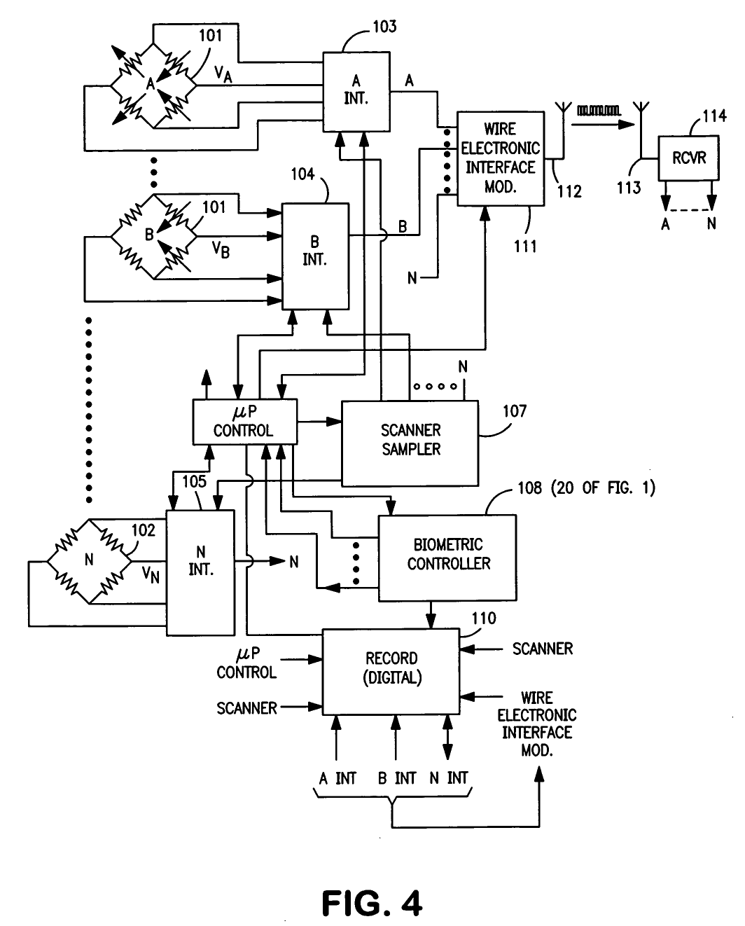

Referring to FIG. 1 there is shown a system according to the present invention. Basically the medical system depicted in FIG. 1 employs wireless transmission for providing sensory signals from a prosthetic or medical device. If reference is made to FIG. 1 it is noted that there are four major components associated with the system. There is a prosthetic device 10 which is operated by a biometric controller 20, where the prosthetic device 10 is associated with a transmitter 40 which transmits a wireless signal to a receiver 50. The transmitter and receiver operate and communicate with each other via a wireless link. As one can ascertain from the Background of Invention and the above noted cited art, biometric controllers 20 have been developed in the prior art and basically generate control signals according to predetermined motion of controlled muscles from the body of the user. In any event, it is also known that a handicapped person, such as a blind person, for example, by receiving or having the ability to depict light without viewing a scene has advantages over a totally blind person. Thus a blind person who can see light without seeing images would know the location of a window or a door or would presumably be able to tell the difference between night and day, while a totally blind person cannot. It has been determined that handicapped users who receive additional information and are familiar with the operation that they are trying to perform will experience much greater flexibility and usage of prosthetic devices upon the receipt of more information. It is difficult, according to prior art techniques, to provide such information in an efficient and reliable way and it is also difficult to provide a great deal of such information without encountering a clumsy and difficult system to maintain and to operate. Again, referring to FIG. 1 there is shown a prosthetic hand, and the prosthetic hand typically has appendages as 18, 11, 12 and 13. Each appendage can be operated by control motors or control devices which are located in housings 15 and 16. The hand will also have a wrist control operated by a wrist control system 17, which wrist control system as well as the appendages are all operated by signals from the biometric controller 20. These signals from the biometric controller 20 are sent to the various motors via the output leads such as 23, 24 and 25. Although three output leads are shown, there can be many more as is understood in the prior art. The biometric controller receives input signals directly from the user, which input signals are generated by sensors associated with a moving part of the user's body. It is also understood that such biometric control signals can be generated by a computer, where the user selects a desired prosthetic movement and the computer generates signals to the prosthesis control motors. For example of such techniques references made to the above noted patents, and particularly reference is made to U.S. Pat. No. 5,480,454 which shows control systems for prosthetic devices and which indicate and provide biometric control. The biometric controller 20 produces output signals according to the controlled movement of a body part from the user as indicated by inputs 21 and 22. It is also indicated that while two inputs are shown there maybe more inputs. The biometric controller, as will be explained, also has outputs as 36 and 37 which are directed to the receiver 50 and outputs 38 and 39 which are directed to the transmitter 40. As will be explained, the transmitter produces an output signal which is transmitted via the antenna 45 to the antenna 60 of the receiver module 50. The output signal from the transmitter enables the receiver 50 to produce electronic signals or output voltage signals which are then connected via electrodes to the nerves or various muscles of the user's body. These output leads are designated as 51 and so on, as will be further explained. According to the system and present invention, distributed throughout the prosthetic hand are clusters of sensors as sensors 25, 39 and 32 associated with appendage 18. These sensors, as will be described, may be stress or pressure sensors and produce outputs according to the pressure or force applied by the appendage 18 during operation, as for example, lifting or otherwise positioning a device or turning a doorknob or various other movements that are typical with the movement of the hand. Thus shown located on each appendage are a cluster of sensors, such as appendage 11 has top sensors 14 and 30, central sensors 35 and bottom sensors 36. As indicated, each appendage has sensors located on the top center and bottom portions of the appendages which basically operate as fingers. Such devices are employed in the prior art and are controlled by motors, hydraulics or other control devices as controlled by the biometric controller 20 as indicated by the above-noted references cited in the Background of Invention. The prosthetic hand also has a wrist portion which is controlled by a motor 17 and to receive signals on output 25. This section is also associated with clusters of sensors as 33 and 34, as well as the distribution of other sensors. In any event, each of the sensors produce outputs proportional to force or proportional to strain as applied to the area during operation. The output of each sensor is coupled to an input as 41 and 41N of the transmitter module 40. This transmitter module, as will be explained, develops an output signal which is transmitted to the receiver module 50. The transmitter also has buttons or switches such as 41, which buttons or switches enable the user to select various sensor clusters during the use of the device. While a single switch 41 is shown, there can be multiple switches, such switches are extremely small and can be located on a pad or other device associated with the transmitter. It is noted that the transmitter is located on or near the prosthetic device and may be on the top surface of the prosthetic or may be located in the hollow of the prosthetic device with the switches being located on top surface. The switches are small and, as indicated, can be operated by a pin or other device, and such switches are used in watches and other digital devices and are well known. In regard to the above and as indicated, the sensors on each of the appendages, as well as the wrist, are arranged in clusters. What is meant by the word “cluster” is that each area may contain one or more sensors and may contain as many as twenty sensors, which would be distributed about the area and which outputs would be directed to processing circuitry associated with the transmitter 40 as will be explained. Thus, basically and as seen from FIG. 1, the prosthetic hand, including the appendages as 11, 12, 13 and 18 are manipulated or moved by the biometric controller which controls motors or other devices 16 associated with the housing section 15, as well as for example, controlling the wrist portion 17 associated with sensor clusters 33 and 34. Thus during operation the transmitter produces a signal which is coupled to antenna 45 and transmitted as evidenced by the transmit signal to the antenna 60 associated with receiver 50. The receiver 50 has multiple outputs such as 51, 52, 55, 56 and so on, which outputs provide voltages which are coupled to electrodes which are placed through the skin of the user or permanently connected to nerves in the arm or elsewhere of the user to enable the user to receive electrical nerve stimulation from the process signals according to the movement of the prosthetic device. It also will be explained that based on the function of the biometric controller 20, that the system may operate to provide the transmission of only selected cluster sections, according to the operation to be performed by the prosthetic device as determined by the biometric controller. For example and as one can ascertain, a human finger has top section, a middle section and a bottom section. These sections will operate differently depending on a task to be performed. Thus, when turning a knob or a dial, mainly the top section of a finger and the thumb may be employed. In other operations such as firmly holding a pencil or other device, other finger sections may be employed, this includes wrist operation as well. Thus the biometric controller produces signals according to the control afforded by the user who operates or energizes various different muscle groups, or utilizes a computer or presses buttons to afford a desired action. Thus based on the predetermined operation of the biometric controller, clusters of sensors can be selected instead of activating all sensors. Thus as shown in FIG. 1 for example, during a certain operation, cluster 25 of appendage 18 may be operated together with cluster 14, 30 and 35 of appendage 11, as well as cluster 33 and 34 associated with wrist section 17. While a number of clusters are shown, it is understood that many other clusters, as for example, many other sensors may be employed in different areas and be utilized accordingly. It is also noted that transmission between the transmitter 40 and receiver 50 is implemented utilizing a wireless technique. According to such techniques, reference is made to U.S. Pat. No. 7,283,922 entitled Transducer Employing Wireless Transmissions for Sending and Receiving Signals issued on Oct. 16, 2007 to A. D. Kurtz, et al. and assigned to Kulite Semiconductor Products, Inc. That patent shows a transducer which operates with transmitted frequency signals. Essentially the signals are sent from a monitoring station to a tuned antenna and then transmitted to a wireless electronic interface or receiver which is associated with a transducer. The patent describes the frequency band which is used and it is desirable, as in this instance, to use a UHF frequency band because it is an unregulated band and operates around 400 MHZ or 900 MHZ. The antennas utilized in this range are very small and signal propagation is not affected by humidity or other disturbances. The above noted patent is incorporated herein in its entirety and basically shows that the wireless transmission between a transmitter 40 and a receiver 50 is easily accommodated. As indicated above, the system preferably utilizes semiconductor sensors which basically are well known, and many of which are manufactured by Kulite Semiconductor Products, Inc., the assignee herein. Kulite has many patents regarding piezoresistor sensors which devices can be employed herein. It is noted that while piezoresistors sensors are preferred, that any type of sensor or strain gage can be utilized according to the teachings of this invention. Such devices, apart from semiconductor devices may be larger and therefore a lesser number may be positioned in the various cluster areas as shown, thus semiconductor devices are preferred.

Referring to FIG. 2 there is shown in FIG. 2A a typical semiconductor sensor, the sensor basically has a silicon cuplike structure 60 which has a central active area 62 which operates as a deflecting diaphragm. Located upon the active or deflecting area 62 are sensors 63 and 64 which are shown. The substrate 60 is typically bonded to a glass supporting wafer 61. Referring to FIG. 2B there is shown a Wheatstone bridge arrangement comprising piezoresistors 65, 68, 66 and 67 which correspond to resistor 63 and 64 of FIG. 2. It is seen that the Wheatstone bridge is biased by applying a voltage (+) to one terminal and a voltage (−) to the other terminal. It is of course understood that one of the terminals may be grounded and therefore the voltage would be applied to the bridge. The bridge, as understood, produces an output proportional to an applied force, stress or pressure and the output is seen as an analog output. It is also noted that while an analog output is shown, that the analog output, as will be further explained, can be converted to a digital signal by a means of an analog to digital converter, which devices are well known. Thus a digital signal can modulate the carrier signal and be transmitted to the receiver which would process the signal and which receiver would have a digital to analog converter to convert the transmitted digital signal to an analog signal for receiver operation.

Referring to FIG. 3 there is shown a general schematic indicative of the cluster concept as described above. As seen in FIG. 3 the so-called thumb appendage, which would be analogous to appendage 18 of FIG. 1, has a top section 71 which would include sensor cluster 25, a middle section 72 which would include sensor cluster 39, and bottom section 73 which would include sensor cluster 32. It further has an ALL section 74 which, if selected, would select all sensors in the thumb cluster 70 and therefore, if the ALL section is selected, then one would select sensors 25, 39, 32 indicated as “T” for top, “C” for center and “B” for bottom. Also shown is a switch 86 which can be mounted on the transmitter and which when operated would select all sensors and thereby when operated manually would automatically select all sensors. The select signals to the top, center and bottom are generated by a microprocessor. In a similar manner there is shown a module entitled finger clusters which basically include appendages 11, 12 and 13, all of which have a top sensor cluster, a central sensor cluster, and a bottom section. In this manner, for appendage 11, when the top module 16 is selected, sensor 14 would be scanned, the center section would include sensor 35 with the bottom section including sensor 36. When one operates the ALL switch 79, all sensors associated with appendage 11 are selected as 14, 35 and 36. The select switch associated with the ALL module 79 is not shown, but such a select switch can also be employed. In a similar manner and referring to FIG. 3 there is a wrist cluster section 80. The wrist section includes cluster A which can include for example, cluster 33. Cluster B which can include cluster 34. Cluster C module 83 which can include another cluster as well as cluster D which can include still another cluster. These clusters are not shown in FIG. 1 but they can for example, be positioned on the other side of the wrist as would be conventional. There is also shown module 85 which selects the Nth or the ALL clusters associated with the wrist. It is seen that an individual cluster, such as cluster D for the wrist can also be selected by the switch 87 which would be closed by the user and cause the sensors associated with module 84, module D to be selected. It is noted that each of the clusters or modules as shown can be manually selected by a switch according to the preference of the user when the user becomes more familiar with the device. It is also understood that giving the user the multiple signals enables him to learn and understand what the signals do by actually noting prosthesis operation or otherwise responding to device control.

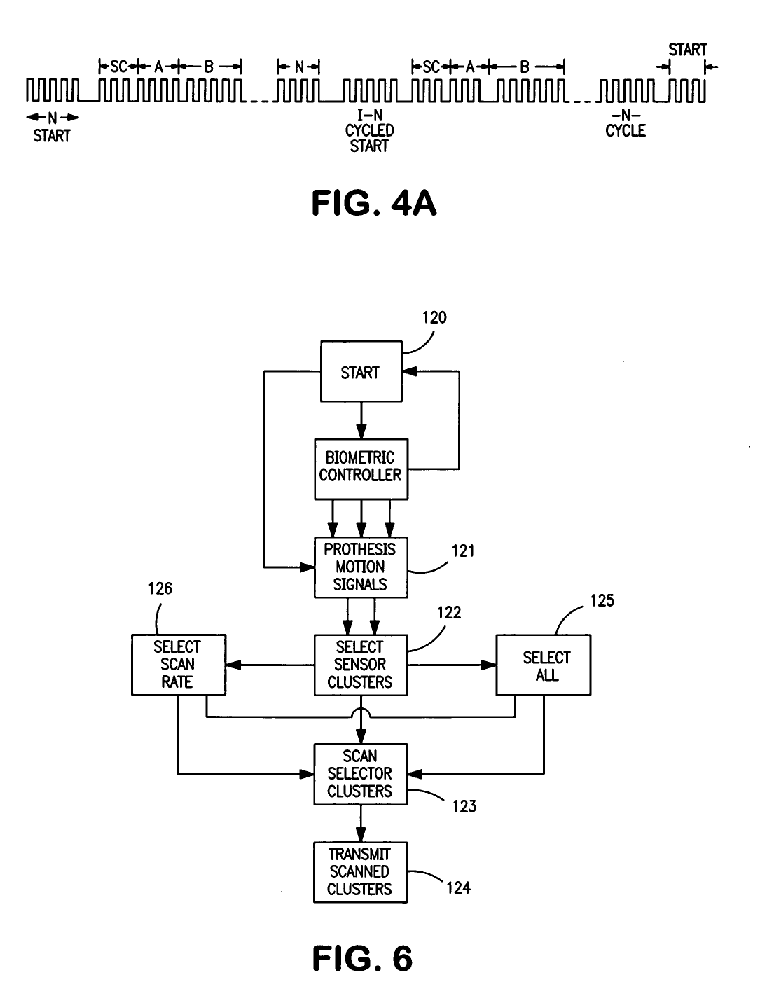

Referring now to FIG. 4A there is shown a typical signal generated by the transmitter as will be further described. Essentially the signal has a start signal which is a predetermined number of pulses or cycles may be a predetermined digital number indicating to the system that transmission has started. The start signal is followed by a delay and then a select cluster signal designated as “SC” is transmitted. The select cluster signal tells the receiver which clusters have been selected at the transmitter so that the receiver can properly respond. This select cluster signal can also include the ALL signal, which means accept all clusters. The start cluster signal is followed by an “A” signal which is indicative of the “A” sensor output followed by a “B” sensor signal indicative of the “B” sensor output and at last the “N” sensor signal indicative of the “N” sensor output. It is understood that there are multiple sensors as A,B,C,D,E, . . . N and so on. The signal proceeds again with another start signal indicating that another signal depicting the selection of various transducers or sensors is being sent. The signal again has a SC signal which is select cluster signal which may be a different signal than the signal first sent. As a select cluster signal, as will be explained, is indicative of the output of the biometric controller which controls the prosthesis device which again is followed by the A signal, the B signal and finally the N signal whereby the signal starts over again with another start signal, as indicated. As one can see, this signal is a continuous transmitted signal which emanates from the transmitter and is generated at the transmitter, as will be explained. Thus the signal contains multiple transducer outputs and for example, may contain hundreds of outputs or more from the various clusters positioned on the prosthetic device. As indicated above, the transmitted signal or the carrier frequency as depicted, may be in the range between 400 MHZ or higher or in the 900 MHZ band. Referring to FIG. 4 there is shown a partial schematic input diagram of a transmitter as transmitter 40 in FIG. 1 and according to this invention. Referring to FIG. 4 there is shown transducers 100, 101 and 102 designated as A, B and N. In a typical system as shown in FIG. 1, transducers or sensors are positioned on the prosthetic device in clusters, as for example some at the top of a finger appendage, some at the center, some at the bottom, some at the wrist and so on. For example and typically there may be a hundred or more sensors. These sensors as indicated, are Wheatstone bridges as shown, and produce a voltage output. Each sensor is associated with a sensor interface as the A interface 103 for sensor A, the B interface 104 for sensor B, and the N interface 105 for sensor N. These interfaces may include compensation devices to compensate the sensors for temperature variations and so on, and also include a switchable amplifier as an operational amplifier, which amplifier can be activated by a scanner sampler 107. The sampler 107 is controlled by microprocessor control circuit 106. Thus each of the interfaces are scanned by scanner 107. The scanner 107, as will be further explained, is controlled by the microprocessor 106, which microprocessor 106 also interfaces with each of the interface circuits as 103, 104 and 105 and thus knows exactly what information is being transmitted and when it is being transmitted. The sensors are selected by means of the microprocessor control 106 which essentially receives information from the biometric controller 108. This information is processed by a microprocessor to determine what function the biometric controller is going to accomplish. The microprocessor then selects the clusters of sensors to be scanned during the operation and also sends a control signal to the scanner or sampler 107 which tells the scanner which units are to be scanned as well as indicating the scanning rate. It is of course understood that the same scanning rate can be used to scan ten sensors as well as to scan a hundred sensors. It is well known that the scanning rate is determined by the Nyquist frequency and scanned at the Nyquist rate so that the proper output would be provided. It is also understood that the microprocessor can change the scanning rate according to the number of clusters or sensors to be scanned and therefore produce a different signal having a larger number of pulses associated with each transducer within the same time limit. In any event it would be desirable to control the scanning rate according to the number of sensors to be scanned in regard to a cluster. The microprocessor control also is coupled to an electronic interface modulator 111. As will be explained, the modulator 111 receives the output from each of the interface units as A, B and N and essentially takes each output and produces a continuous signal by applying each time period to a modulator which modulates each respective time period with a carrier signal and produces an output composite signal which is coupled to antenna 112 which may be, as indicated, in the above noted U.S. Pat. No. 7,283,922 patent a tuned antenna or other device. The composite signal is then transmitted to the receiver, where the signal is received by an antenna 113, which also may be a tuned antenna. The receiver 114 produces the plurality of output signals which signals, as will be explained, are analog signals approximating or proportional to the voltage of each sensor output as sensors 100, 101 and 102. The outputs of the receiver as output A to N are then converted to analog signals where they are then coupled to various nerves or nerve bunches to enable the user to experience sensory sensations according to the strain or pressure experienced by the prosthetic device. Also shown in FIG. 4 is a recorder 110 which recorder 110 is also under control of the microprocessor. Recorder 110 is a digital recorder which will record all output signals such as the microprocessor control signals, the scanner signals as well as the output from the wire electronic interface modulator prior to modulation which would be, for example the outputs A, B and N. This recorder would operate to record signals for later playback and use by the user of the prosthetic device, as these signals, for example would emulate certain control functions of the prosthetic device and enable the user to experience the output of the recorded signals by applying those signals directly to the nerves of the user via the implanted electrodes. It is of course understood that the user while actually using the prosthetic device will feel the voltage signals generated by the system during operation and after a time period will understand exactly what the prosthetic device is doing and therefore, in response to the received signal, have an indication of how much pressure should be applied to the device via the control of the biometric controller 108. It is also understood from the above noted description that this invention is not concerned with biometric control, but has to do with providing signals to a user proportional to the amount of pressure or strain applied by the prosthetic device to a given surface according to the pressure sensor clusters as arranged in predetermined given locations on the prosthesis.

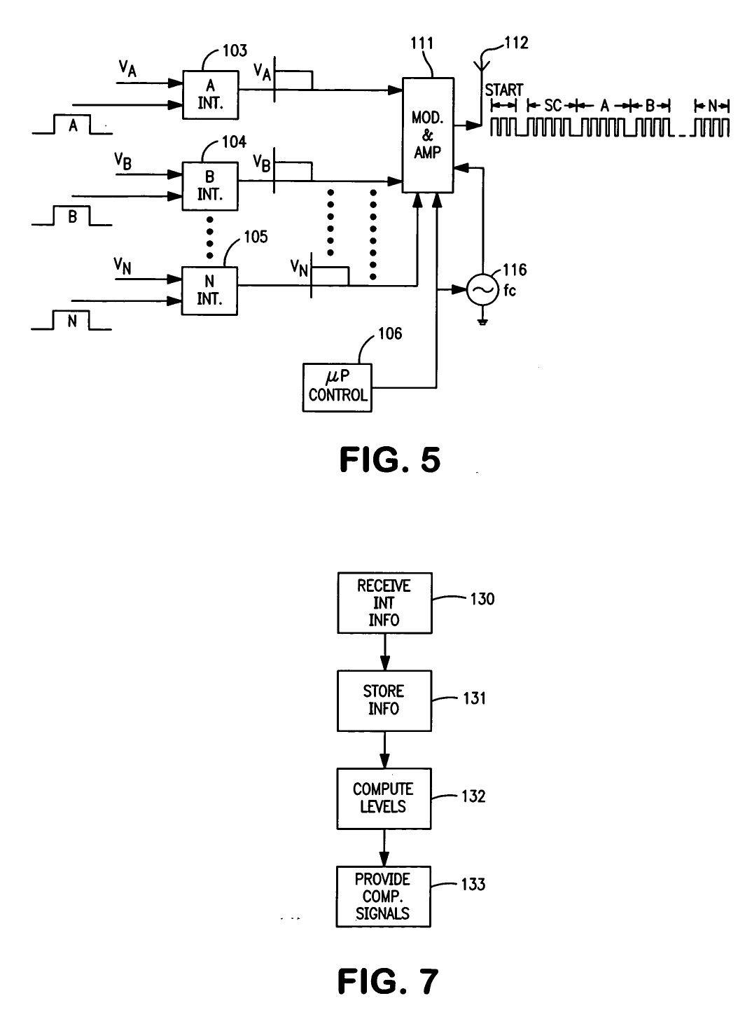

Referring to FIG. 5 there is shown a block diagram of the interface modules as 103, 104 and 105 in FIG. 4 having their outputs coupled to the inputs of the modulator and amplifier 111 and having an output coupled to the antenna 112. As indicated, the scanner or multiplexer produces pulses which activate each interface during the respective time period. The pulses are sampling pulses and essentially are displaced in time according to the sampling rate. Thus the outputs are shown in FIG. 5 and when coupled to the input of the modulator, each output is modulated by the carrier frequency and an output signal as shown in FIG. 3 is generated by the modulator. There is shown the carrier oscillator 116 which as indicated, provides a modulation frequency of 400 MHZ or 900 MHZ. Also shown is the microprocessor controller 106, the controller 106 basically provides the modulator with the start interval as well as with the select cluster information and essentially enables that information to be transmitted via the signal output from antenna 112, as described above, so that the receiver is cognizant of the clusters that are pertinent as well as the start sequence so that the scanner or demultiplexer in the receiver can be synchronized accordingly. As indicated above, the carrier signal as applied to the modulator may be frequency, phase or amplitude modulated according to the magnitude of the DC signals applied to the input as the A, B and N signal. Essentially the modulator has multiple inputs, all of which are coupled to the same terminal whereby the step or sampled signal produces the output signal shown in FIG. 4A.

Referring to FIG. 6 there is shown a flow chart of some of the functions that the microprocessor control 106 implements as shown in FIG. 4. According to the flow chart, when the biometric controller which is coupled to the microprocessor provides a signal, the microprocessor begins a start sequence as evidenced by module 120. It is of course noted that the signal contains a start pulse which again, is generated by the microprocessor prior to the transmission of the output signal. The start signal is followed by a spacing, all of which are generated by the microprocessor and sent to the modulator interface 111. The biometric controller which is biometric controller 108 of FIG. 4 or controller 20 of FIG. 1, sends signals to the microprocessor, where the microprocessor as indicated in module 121 samples the prosthesis motion signals as generated by the biometric controller which are sent to the control motors. These signals then are processed by the microprocessor determine the select sensor cluster signal as evidenced by module 122. Thus module 122 depending on the prosthesis motion signals, develop the select cluster signal and essentially sends that signal to the modulator to provide the SC signal shown in FIG. 4. This essentially informs the scanner, as controlled by the microprocessor, which clusters are to be scanned as evidenced by module 123. The scanner, as controlled by the microprocessor then scans the selected signals and the selected signals as scanned are sent to the modulator 111 and then transmitted via the antenna 112 as evidenced by module 24. Also seen coupled to the select sensor cluster module 122 is a module designated as select ALL clusters 125. The module select ALL clusters can be generated by the select sensor cluster module 122. For example, if the prosthetic device is to perform a complicated motion like picking up and putting down a rod, where the entire hand as well as the wrist motion is being implemented and all appendages or fingers are being used, as well as the wrist, then the select ALL sensor module 125 will be activated by the microprocessor and thus all clusters, which include all sensors would be scanned. The microprocessor also will select a scan rate as evidenced by module 126. As indicated above, the sensors may be one hundred or more sensors and the various clusters may be divided into many areas, each of which may contain one or more sensors in a cluster area. Thus if the microprocessor determines that for example only three clusters or three sensors are to be scanned, then the scanning rate can change and therefore instead of scanning all sensors or all sensors in a cluster the change of the scanning rate can provide faster signal transfers while still be scanned at a proper data rate.

Referring to FIG. 7 there is shown another function that the microprocessor performs. As shown in FIG. 4 the microprocessor is also coupled to and receives information from each of the sensor interfaces as the A interface 103 up to the N interface 105. This information is received by the microprocessor as evidenced by module 130. The information is stored in the microprocessor memory as for example, in a random access memory or other peripheral memory as indicated in module 133. The microprocessor when receiving the stored information also has information indicative of the characteristics of each of the sensors. This information can be used by the microprocessor to change the level at each interface module according to temperature as well as according to the particular characteristics of the selected sensor. Such techniques have been developed by Kulite Semiconductor Products, Inc. and essentially enable one to compensate the output of a piezoresistor sensor according to the temperature and so on by a microprocessor eliminating the need to separately trim or correct each sensor. Thus the microprocessor can provide compensating signals for each of the sensors as evidenced by module 133.

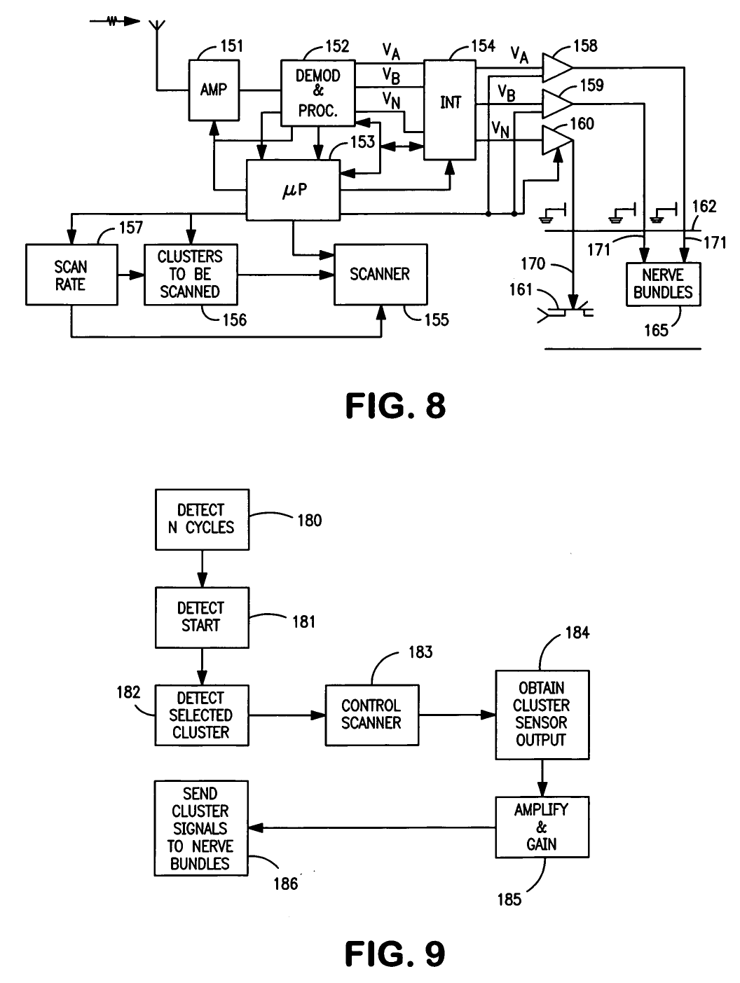

Referring to FIG. 8 there is shown a receiver block diagram. The receiver as depicted in FIG. 8 is analogous to the receiver 50 of FIG. 1 as well receiver 114 of FIG. 4. Essentially as one understands, the antenna 150 receives a transmitted signal from the transmitter. The signal is amplified by an RF amplifier 151. The RF amplifier is controlled in gain by a microprocessor 153. The output from the RF amplifier goes into a demodulator 152. The demodulators are well known and essentially the demodulator serves to take the carrier frequency and strip the carrier frequency from each of the transmitted channels. Thus the demodulator provides outputs which are equivalent to the voltages A, B and N as for example shown in FIG. 5 as the inputs to the modulator 111. The demodulator 152 as will be explained, demodulates the transmitted signal in conjunction with the scanner 155 or demultiplexer to produce output voltage signals A, B and N as the signals shown in FIG. 5 as VA, VB and VN. These signals are directed to an interface which is controlled by the microprocessor as the signals may be further varied in amplitude or in phase by the microprocessor. The voltage signals are applied to DC amplifiers 158, 159 and 160. Each of the A cluster, B cluster and the N cluster have each sensor voltage amplified and the amplified output is applied to probe or needle devices similar to acupuncture needles. The needles as 170, 171 and 173 are placed in position by a physician or therapist. In lieu of using needles one can also use conductive electrodes which are coupled to the amplifier outputs. As seen, the amplifiers produce DC voltages which are proportional to the DC voltages generated by the sensors or probes during each of the scanned intervals. As one can ascertain, the scanner 155 at the receiver operates to enable one to retrieve each of the sensor signals from the transmitted signal. As indicated, the microprocessor 153 receives the demodulated signal and therefore can calculate the start signal and essentially determine that after the start signal the clusters to be scanned, for example, are determined. Once the microprocessor determines which clusters are to be scanned then information is transmitted to the scanner which scanner understands that the clusters, for example A and B are to be scanned as opposed to all clusters. The microprocessor, as indicated by module 157 also determines the scan rate for scanner 155 so that the demodulator can be controlled according to the number of sensors to be scanned. It is also indicated that all sensors may be scanned and the microprocessor will determine this based on information in the transmitted signal which is the select cluster signal SC.

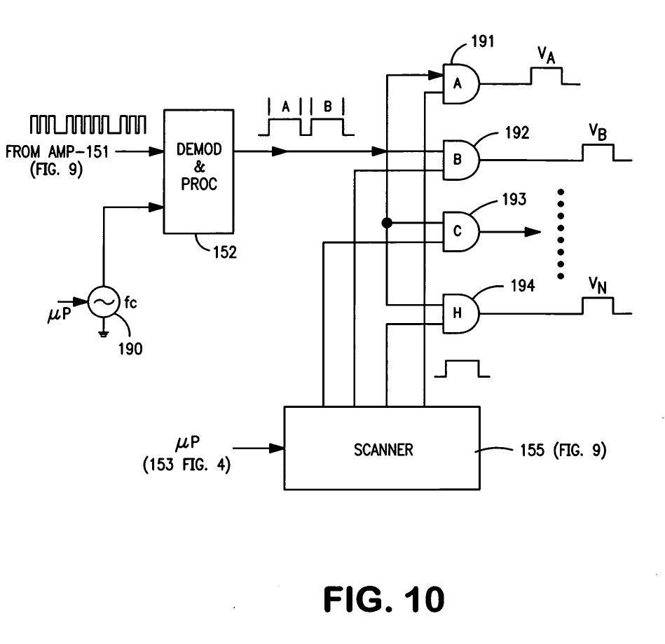

As seen in FIG. 9 there is a flow diagram basically showing receiver microprocessor operation. In reference to FIG. 9 there is shown the microprocessor 153 operation in the receiver. Essentially the microprocessor 153 receives the demodulated transmitted signal from demodulator 152. It then detects the N cycles indicative of the start and therefore detects the start as evidenced by modules 180 and 181 of FIG. 9. In detecting the start the microprocessor expects the select scan signal to follow. The select scan signal tells the microprocessor which sensors were scanned at the transmitting end. Thus the microprocessor now informs the scanner 183 as to which sensors or clusters should be scanned. The scanner thus scans the desired clusters and obtains the cluster sensor DC output as evidenced by module 184. This corresponds to output A, B and N shown in FIG. 8. The cluster sensor output which are DC signals are then amplified or further modified by DC amplifier 158, 159 and 160. These amplifiers can be controlled in gain by the microprocessor, this also is a sub routine and can be programmed by the system provider. In other words, certain signals may have to be amplified in order to produce the proper nerve stimulation. In any event, the signals as emitted by 185 are sent to the nerve bundles and impinge upon the nerves as shown in FIG. 8 as nerve bundles 161 and 165. Thus as shown in module 186 the amplified signals as signals A, B and N are now sent to the DC amplifiers and associated probe as 170, 171 and 173 to apply these signals to the appropriate nerve bundles.

Referring to FIG. 10 there is shown a block diagram of the demodulator and processing depicted in FIG. 8. As seen, the demodulator receives the transmitted signals from amplifier 151 and essentially demodulates the signal employing the oscillator 190. Oscillator 190 produces the carrier signal which is synchronized with the transmitted signal by the microprocessor. In any event, demodulation takes place where the carrier signal is stripped from the transmitted signal and thus the sensor signals appear at the output of the demodulator as signals A, B and so on. The demodulator signals are applied to plurality of output gate devices such as 191, 192, 193 and 194 and so on. Essentially the gate devices receive the demodulated signals at one input, and receive the scanner signal at the other input as from scanner 155. Thus the gates 191, 192, 193 and 194 produce the signals VA, VB and VN which essentially are the A, B and C signals obtained at the output of the interface module 154. The interface module 154 may contain the gates and is under control of the microprocessor. It is of course understood that the circuitry to implement the receiver transmitter is well known, for example, scanners are normally implemented by binary counters or by shift space registers which will produce outputs which step in time and scanning of various modules is also well known. The gating of these signals to produce the outputs VA, VB and VN is also well known as shown in FIG. 10. The entire apparatus for the receiver can be implemented by a DSP or digital signal processor which can be custom built as well as a custom built integrated circuit will implement all circuitry for both the transmitter and the receiver. Thus as one can understand the primary objective of the above noted invention is to provide pressure sensors on a prosthetic device, which sensors can respond to strain or pressure and which provide a signal proportional to an applied pressure or force on the prosthetic device. This signal indicative of applied pressure is responded to by a transmitter. The transmitter converts the signal to a high frequency signal and produces a continuous signal which can be transmitted to receiver. The receiver is placed on the operative part or the uninjured part of the patient's arm or appendage. The receiver has outputs connected to nerves which are then stimulated by the demodulated receiver signals. In this manner by providing a plurality of clusters of sensors, one can scan each cluster according to a desired motion of the prosthetic device and therefore stimulate the nerves of the user according to the selected cluster and according to the controlled movement of the prosthetic device.

While the above noted invention has been shown and described in the various figures, it is apparent to one skilled in the art that many alternative configurations could be employed, all of which are encompassed within the spirit and scope of this invention. It is indicated that the signal transmitted could contain the sensor output information in a form of a phase modulation or frequency modulation. The signal can also be transmitted with amplitude modulation. All such modulation techniques are well known. The scanning can be accomplished by various means including the microprocessor itself which can scan the various sensors and produce the outputs or by a multiplexer. As indicated above the microprocessor can perform many other functions apart from the functions described. Thus as one can ascertain there are many modifications and changes than can be implemented, all of which are deemed to be encompassed within the spirit and scope of the Claims appended hereto.

What is claimed is:

1. A medical system for providing sensory signals to a user indicative of the force or pressure applied by a medical device controlled by the user, comprising:
a plurality of force responsive sensors positioned on said device in predetermined locations, each of said devices operative to provide an output signal proportional to said applied force,
scanning means scanning each of said sensors to provide a plurality of time sequential output signals each indicative of an associated sensor output, and
a transmitter means coupled to said scanning means and operative to transmit a signal to a remote location indicative of said output signal of at least a first selected plurality of said force responsive sensors.

2. The system according to claim 1 further including:
a receiver means located on said user's body having an antenna for receiving said transmitted signal,
processing means coupled to said antenna for providing at an output a plurality of signals, each indicative of the signal value of each sensor in said selected plurality, and
means coupled to said predetermined nerve areas of said user to stimulate said nerve area according to at least one of said sensor signal values.

3. The medical system according to claim 1 wherein said medical device is a prosthesis.

4. The medical system according to claim 3 wherein said prosthesis is a hand prosthesis having at least two finger appendages each having a top section, a center section and a bottom section.

5. The medical system according to claim 4, further comprising:
a first cluster of force responsive sensors positioned on the top section of said first appendages,
a second cluster of force responsive sensors positioned on the middle section of said first appendage,
a third cluster of force responsive sensors positioned on the bottom section of said first appendage,
said second appendage having third, fourth and fifth force responsive sensor clusters respectively positioned on said appendage near the top, center and bottom.

6. The medical system according to claim 5, further including a biometric controller operative by said user to cause said prosthesis to exhibit a desired move and for providing biometric output signals indicative of said move,
a processor coupled to said biometric controller for receiving said output signals to provide a scanning control output indicative of which clusters of said force responsive sensors are to be scanned, said scanning control output coupled to said scanning means to control the scanning sequence accordingly.

7. The medical systems according to claim 5 wherein each cluster may have between 1 and 20 force responsive sensors.

8. The medical system for transmitting force signals imposed by a prosthetic device during movement and use of said device by a user, said transmitted signals received by a receiver located on the body of said user, comprising:
a prosthetic device having at least one surface for imparting a force to an object during use,
a plurality of force responsive sensors located in predetermined positions on said surface, each sensor capable of providing an output proportional to a force applied thereto,
scanning means coupled to said sensors and operative to scan each sensor during a predetermined period to provide output signals each indicative of each scanned sensor output,
modulation means responsive to said output signals to provide a composite signal for transmission to a remote location,
receiving means located on the body of said user and operative to receive said corporate signal to provide output signals indicative of each scanned sensor output, and
means responsive to said output signals to apply each output signal to a preselected nerve area of said user to enable said user to receive sensory feedback pertinent to the use of said prosthetic device.

9. The medical system according to claim 8 wherein said prosthesis is a hand prosthesis having at least two finger-like appendages, with each appendage having a first sensor cluster at the top of said appendages, a second sensor cluster at the center of each appendage and a third sensor cluster at the bottom of said appendage.

10. The medical system according to claim 9 wherein said scanning means includes means for selecting clusters of sensors to be scanned on said first and second appendages according to a predetermined use of said prosthetic device.

11. The medical system according to claim 8 wherein said force responsive sensors are piezoresistive sensors.

12. A method of providing sensory feedback signals to a user of a prosthetic device comprising the steps of:
placing force responsive sensors on moveable areas of said prosthetic device, each of said sensors capable of providing an output according to a force applied by said prosthetic device to an apparatus,
arranging and modulating said outputs in a serial format with each sensor having an allocated times slot, to provide a transmission signal,
transmitting said signal
receiving said transmitted signal
arranging and demodulating said transmitted signal to provide a plurality of individual sensor output signals and
applying said individual sensor output signals to individual associated nerve areas of said user to stimulate said nerve areas according to the operation of said prosthetic device.

13. The method according to claim 12 wherein the step of arranging and modulating includes the step of scanning said force responsive sensors in sequence and applying said scanned sequence to a modulator for transmission.

14. The method according to claim 12 further including the step of:
selecting only certain said sensors to be scanned according to a desired movement of said prosthetic device.

15. A medical system employing wireless transmission for providing selective feedback to a handicapped person using a medical aid, comprising:
at least one sensor coupled to said medical aid and operative to provide an output signal according to a condition of said medical aid,
a transmitter responsive to said output signal to transmit said signal to a remote location,
a receiver located at said remote location and operative to receive said transmitted signal to provide sensor output signal,
means responsive to apply said signal to a selected nerve area of said handicapped person to stimulate said nerve area according to the magnitude of said output signal.

16. The medical system according to claim 15 wherein said medical aid is a prosthesis having moveable parts which parts are manipulated by said handicapped person using a biometric controller, further comprising:
a plurality of force sensors positioned on said moveable parts in predetermined areas and arranged in a cluster,
selection means responsive to signals from said biometric controller to select a least one cluster of force sensors to be addressed,
scanning means responsive to said selected cluster to scan said sensors in said selected cluster to provide a plurality of output signals, one signal output for each sensor in said cluster,
modulation means for modulating said sensor signals to provide an output signal for transmission to a remote location.

17. The medical system according to claim 16 wherein said selection means is operative to provide a select cluster signal to inform said receiver as to which clusters were scanned and means for including said select cluster signal in said output signal to be transmitted.

18. The medical system according to claim 17 wherein said force sensors are piezoresistive semiconductor devices.

19. The medical system according to claim 18 wherein said transmitted signal is in the UHF band.

20. The medical system according to claim 16 further including a microprocessor coupled to said biometric controller and said scanning means and operative to control said scanning means according to a cluster selected by said microprocessor as determined by said biometric controller signals.
